(19) United States
(12) Patent Application Publication
(10) Pub. No.: US 2005/0014849 A1
(43) Pub. Date: Jan. 20, 2005

(54) Antifungal phenylethylene

(76) Inventors: George R. Pettit, Paradise Valley, AZ (US); Robin K. Pettit, Fort McDowell, AZ (US)

Correspondence Address: Fennemore Craig, Suite 2600, 3003 North Central, Phoenix, AZ, 85012-2913, US

(21) Appl. No.: 10/499,958

(22) PCT Filed: Dec. 20, 2002
(86) PCT No.: PCT/US02/40930

(30) Foreign Application Priority Data
Dec. 22, 2001 (US) 60343067

(51) Int. Cl. A61K 31/075

(52) U.S. Cl. 514/716

(57) ABSTRACT

The antifungal and cancer cell growth inhibitory activities of 1-(3′,4′,5′-trimethoxyphenyl)-2-nitro-ethylene (TMPN) were examined. TMPN was fungicidal for the majority of 132 reference strains and clinical isolates tested, including those resistant to fluconazole, ketoconazole, amphotericin B or flucytosine. Minimum fungicidal concentration/minimum inhibitory concentration (MFC/MIC) ratios were ≦2 for 96% of Cryptococcus neoformans clinical isolates and 71% of Candida albicans clinical isolates. TMPN was fungicidal for a variety of other basidiomycetes, endomycetes and hyphomycetes, and its activity was unaffected by alterations in media pH. TMPN was slightly cytotoxic for murine and human cancer cell lines (GI₅₀=1-4 μg/ml), and weakly inhibited mammalian tubulin polymerization (IC₅₀=0.60 μg/ml). TMPN may also be used as a biochemical probe for tubulin and fungal dimorphism studies.

### RELATED APPLICATION

This application claims the priority of U.S. Provisional Application No. 60/343,067, filed Dec. 22, 2001, entitled “Antifingal Phenethylene,” which is incorporated herein by reference.

### GOVERNMENT INTEREST

This invention was funded in part by the NIH OIGCA44344-01-011. The United States Government may have certain rights in this invention.

### INTRODUCTION

This invention relates to phenethylene compounds having antifungal activity. More particularly this invention relates to the development of a phenethylene having significant antifungal activity, namely 1-(3′,4,′5′-trimethoxyphenyl)-2-nitro-ethylene, which may also be used as a biochemical probe for tubulin and fungal dimorphism study.

### BACKGROUND

The major classes of antifungal drugs available for clinical use are the macrolide polyenes, fluoropyrimidines, azoles and the allylamines/thiocarbamates [1]. These agents are limited by toxicity, fungistatic mechanisms, narrow activity spectra and/or drug resistance [1]. The limited selection of effective antifungals, combined with the emergence of previously uncommon fungal pathogens [2] and an increasing population of immunocompromised patients, has resulted in a critical need for new antifungal agents. The development of compounds of novel structural class that have a fungicidal mechanism and a broad spectrum of activity will likely have the greatest impact on the current crisis.

### BRIEF DESCRIPTION OF THE INVENTION

A recent review of the antifungal actions of antineoplastic agents concluded that antineoplastic agents and their derivatives are an excellent resource for the discovery of novel antifungal targets and agents [3]. A lead in vitro antifungal compound with cancer cell line inhibitory activity, 1-(3′,4′,′5′-trimethoxyphenyl)-2-nitro-ethylene (TMPN) was discovered. TMPN was synthesized as part of a structure/activity study of trimethoxybenzene antitubulin compounds like podophyllotoxin.

The antifingal and cancer cell growth inhibitory activities TMPN were examined. TMPN was fungicidal for the majority of 132 reference strains and clinical isolates tested, including those resistant to fluconazole, ketoconazole, amphotericin B or flucytosine. Minimum fungicidal concentration/minimum inhibitory concentration (MFC/MIC) ratios were ≦2 for 96% of Cryptococcus neoformans clinical isolates and 71% of Candida albicans clinical isolates. TMPN was fungicidal for a variety of other basidiomycetes, endomycetes and hyphomycetes, and its activity was unaffected by alterations in media pH. The frequency of fungal spontaneous mutations to resistance was <10⁻⁶.

Kill curve analyses confirmed the fungicidal action of TMPN, and demonstrated that killing was concentration- and time-dependent. At sub-MIC exposure to TMPN, C. albicans did not exhibit yeast/hyphae switching. TMPN was slightly cytotoxic for murine and human cancer cell lines (GI₅₀=1-4 μg/ml), and weakly inhibited mammalian tubulin polymerization (IC₅₀=0.60 μg/ml). The in vitro profile of TMPN warrants its development both as an in vivo antimicrobial for superficial and cutaneous mycoses, and as a biochemical probe for tubulin and fungal dimorphism study.

### BRIEF DESCRIPTION OF THE DRAWING

FIG. 1. Structure of 1-(3′,4′,5′-trimethoxyphenyl)-2-nitro-ethylene.

FIG. 2. Kill curves for C. neoformans ATCC 90112 (A) and C. albicans ATCC 90028 (13) with indicated multiples of the 1-(3′,4′,5′-trimethoxyphenyl)-2-nitroethylene MIC. Results are means±the standard errors of the means.

FIG. 3. Percentage of C. albicans ATCC 90028 cells with buds (solid lines) or hyphal extensions (dotted lines): control cells treated with DMSO (squares); cells treated with one quarter times the TMPN MIC (upside down triangles); cells treated with one half times the TMPN MIC (circles). The results are presented as means±standard errors of the means.

FIG. 4. Morphological characteristics of control and TMPN-treated C. albicans ATCC 90028 observed with video-enhanced DIC optics. Scale bar=10 μm. In panel a, yeast growth (arrowhead) and hyphal growth (arrows) morphologies were observed under control growth conditions. In panel b, only yeast growth (arrowheads) morphology was observed in cultures treated with one half times the TMPN MIC.

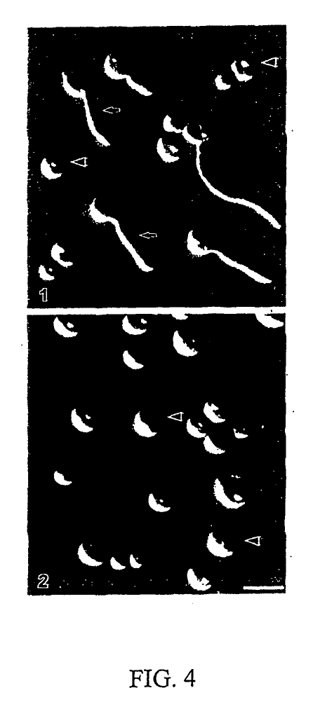

### DETAILED DESCRIPTION OF THE PREFERRED EMBODIMENT

The major classes of antifungal drugs available for clinical use are the macrolide polyenes, fluoropyrimidines, azoles and the allylamines/thiocarbamates [1]. Toxicity, fungistatic mechanisms, narrow activity spectra and/or drug resistance [1] limit these agents. The limited selection of effective antifungals, combined with the emergence of previously uncommon fungal pathogens [2] and an increasing population of immunocompromised patients, has resulted in a critical need for new antifungal agents. The development of compounds of novel structural class that have a fungicidal mechanism and a broad spectrum of activity will likely have the greatest impact on the current crisis.

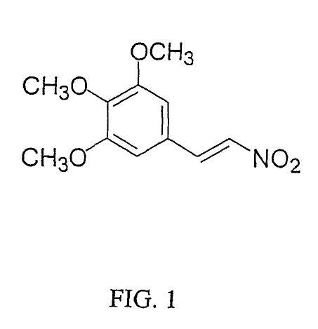

A recent review of the antifungal actions of antineoplastic agents concluded that antineoplastic agents and their derivatives are an excellent resource for the discovery of novel antifungal targets and agents [3]. The present invention demonstrates the in vitro development of an antifungal compound with cancer cell line inhibitory activity, 1-(3′, 4′,5′-trimethoxyphenyl)-2-nitro-ethylene (TMPN) who's structural formula is depicted in FIG. 1.

TMPN was synthesized as part of a structure/activity study of trimethoxybenzene antitubulin compounds like podophyllotoxin. The effect of TMPN on a variety of cell types was investigated. The in vitro profile of TMPN warrants its development as an antimicrobial for superficial and cutaneous mycoses, and as a biochemical probe for tubulin and fungal dimorphism studies. TMPN was synthesized as previously described in the art. TMPN was reconstituted in a small volume of sterile dimethylsulfoxide (DMSO) and diluted in the appropriate media immediately prior to susceptibility experiments.

Reference strains were obtained from the American Type Culture Collection (Rockville, Md.) or Presque Isle Cultures (Presque Isle, Pa.). Strains were maintained by single colony transfer on nutrient agar at 35° C. (exceptions were Neisseria gonorrhoeae on gonococcal typing agar [5] at 37° C. with 5% CO₂, and Streptococcus pneumoniae on tryptic soy agar with 5% sheep blood at 37° C. with 5% CO₂.

Nonduplicate clinical isolates were obtained at the University of Virginia Health System. Fluconazole-resistant clinical isolates [Jessup, C. J., Wallace, T. L. & Ghannoum, M. A. (1997) Evaluation of antifungal activity of nyotran against various pathogenic fungi. Poster #F-88. Toronto: 37th Interscience Conference on Antimicrobial Agents and Chemotherapy] were provided by the Center for Medical Mycology, Case Western Reserve University. Reference strains were obtained from the American Type Culture Collection.

Most yeast strains were maintained by single colony transfer on Sabouraud Dextrose Agar (SDA), pH 5.6 at 35° C. Cryptococcus albidus, C. laurentii and C. uniguttulatus (#66033) were maintained on SDA, pH 6.6 at 25° C., Filobasidium uniguttulatum, Kluyveroinyces spp., Trichosporon spp., Blastoschizomyces capitatus, Epiderinophyton floccosum and Paeciloinyces lilacinus on Emmon's modification of SDA at 30° C., and C. uniguttulatus (#34143) and C. ater on Yeast Morphology (YM) agar at 25° C. Rhizopus spp. and Aspergillus spp. were maintained on Potato Dextrose Agar (PDA) slants at 35° C.

Antimicrobial activity was assayed by National Committee for Clinical Laboratory Standards (NCCLS) disk susceptibility tests. Isolated colonies from overnight cultures were suspended and diluted as recommended to yield approximately 1-2×10⁸ cfu/ml, and 50 μl of this preparation immediately spread on agar plates. NCCLS recommended agar media [5] were used for S. pneumoniae and N. gonorrhoeae, and Mueller-Hinton agar for all other bacteria. Yeast strains were tested on SDA. Excess moisture was absorbed for 10 min prior to application of 6 mm paper discs containing two-fold dilutions of TMPN in sterile DMSO. The MIC was defined as the lowest drug concentration resulting in a clear zone of growth inhibition around the disc after 18 h (all organisms except Micrococcus, Candida, Cryptococcus) or 42 h (Micrococcus, Candida, Cryptococcus).

The antibacterial activity of TMPN was also assessed by the NCCLS broth macrodilution assay [6]. Isolated colonies from overnight cultures were suspended and diluted as recommended to yield final inocula of approximately 5×10⁵ cfu/ml. Tests were performed in sterile plastic tubes (12 by 75 mm) containing twofold dilutions of TMPN in gonococcal typing broth (Neisseria), Mueller Hinton II (MHII) (cation adjusted) broth containing 3% lysed horse blood (Streptococcus) or MHII broth (all other bacteria). One tube was left drug-free (but contained an equivalent volume of DMSO) for a turbidity control. Tubes were incubated without agitation at 37° C. with 5% CO₂ (Streptococcus, Neisseria), or at 35° C. (remaining bacteria). MICs were determined after 24 h for all bacteria except Micrococcus, which was read at 48 h. The MIC was defined as the lowest concentration of drug that inhibited all visible growth of the test organism (optically clear).

TMPN was screened against yeasts by the broth macrodilution assay according to the NCCLS [7]. Yeasts were suspended and diluted as recommended to yield final inocula ranging from 0.5-2.5×10³ cfu/ml. Tests were performed in sterile 12 by 75 mm plastic tubes containing two-fold dilutions of TMPN in 0.165 M morpholinepropanesulfonic acid buffered RPMI 1640 medium (pH 7.0). One tube was again left drug free (but contained an equivalent volume of DMSO) for a turbidity control. Tubes were incubated without agitation at the appropriate temperature (see Fungal Strains section above). MICs were determined after 72 h for Cryptococcus and after 48 h for other yeast genera. The MIC was defined as the lowest concentration of TMPN that inhibited all visible growth of the test organism (optically clear).

Susceptibility testing of filamentous fungi was also conducted. Broth macrodilution susceptibility testing of P. lilacinus, Rhizopus spp. and Aspergillus spp. was performed in accordance with a proposed standardized procedure [8] with slight modification. To induce conidium and sporangiospore formation, fungi were grown on PDA slants at 35° C. for 6 days (P. lilacinus, Rhizopus spp., all Aspergillus species except A. nidulans) or 3 days (A. nidulans). Fungal slants were covered with sterile 0.85% NaCl (P. lilacinus, Rhizopus spp., all Aspergillus species except A. nidulans) or 0.05% Tween 80 (A. nidulans), and suspensions were made by gently probing the colonies with the tip of a sterile Pasteur pipette. The resulting mixture of hyphal fragments and conidia or sporangiospores was withdrawn and transferred to a sterile clear microcentrifuge tube, and heavy particles were allowed to settle for 10 min. The upper homogenous suspension was transferred to a sterile microcentrifuge tube, vortexed for 15 s, adjusted spectrophotometrically, and diluted in sterile 0.165 M MOPS-buffered RPMI medium, pH 7.0, to yield final inocula ranging from 0.5-2.5×10³ cfu/ml. Susceptibility to TMPN was then determined by broth macrodilution as described above for yeast isolates. MICs were read after 48 h The MIC was defined as the lowest concentration of TMPN that inhibited all visible growth of the test organism (optically clear).

Minimum fungicidal concentrations (MFCs) were determined by subculturing 0.1 ml from each tube with no visible growth in the MIC broth macrodilution series onto the appropriate drug-free plates (see Fungal Strains section above). The plates were incubated for 48 h, and the MFC was defined as the lowest drug concentration that completely inhibited growth on plates.

Possible host effects were also evaluated. Broth macrodilution assays were performed with RPMI medium prepared at pH 5, pH 6, and pH 7, and in RPMI medium with and without 50% normal human serum (Lampire Biological Labs). The pH experiments were performed twice on separate days.

Determination of the frequency of occurrence of spontaneous mutants was performed as previously described [9]. Overnight cultures of C neoformans (ATCC 90112), C. albicans (ATCC 90028) and Trichosporon inkin (ATCC 18020) were diluted to an OD_{530 nm=}0.3. 0.1 ml of each preparation was spread onto SDA plates containing four or eight times the broth macrodilution MIC of TMPN. The starting inoculum for each organism was also diluted and plated onto drug-free SDA plates for determination of cfu/ml. After a 48 h incubation at the appropriate temperature (see Fungal Strains section above), the number of colonies on drug-supplemented SDA was counted. The frequency of occurrence of spontaneous mutants was calculated by dividing the number of colonies on drug-containing plates by the number of colonies in the inoculum. When no colonies were visualized on drug-containing plates, the calculation was (<) 1 colony divided by the number of colonies in the inoculum.

Time—kill studies were also performed. The proposed standardized procedures of Klepser et al [10] were followed. Overnight cultures of C. neoformans (ATCC 90112) and C. albicans (ATCC 90028) in pH 7.0 MOPS-buffered RPMI 1640 medium were inoculated into the same medium containing multiples of the broth macrodilution MIC of TMPN or an equivalent volume of DMSO. Cultures were shaken at 35° C., and aliquots aseptically removed at various times for dilution plating. In addition, 100 μl aliquots were plated directly from drug-treated flasks at each time point. Thus, the detection limit in these experiments was 10 cfu/ml. Standard errors of the means were calculated from at least two experiments.

The mechanism of action of TMPN was investigated microscopically, Candida albicans (ATCC 90028) cultures were exposed to one quarter or one half times the broth macrodilution MIC of TMPN in DMSO, or an equivalent concentration of DMSO for controls, until late-log phase. Cells were examined using an Axioscope microscope (Carl Zeiss, Thornwood, N.Y.) equipped with standard differential interference contrast (DIC) using a Plan-Neofluar 100×/1.3 (oil immersion) objective. The microscope was coupled to a C24007-07 (imaging tube camera type) video camera, via a 4×extension tube (Carl Zeiss), and an analog control unit (Hammamatsu Photonic Systems Corp., Bridgewater, N.J.). Real-time digital contrast enhancement was done with an Argus 10 Image Processor (Hammamatsu). Single frame images were digitized directly or from videotaped sequences using a Sony UP-5600MD video/digital printer (Sony Electronics, Inc., Montvale, N.J.) and prepared for printing in Photoshop 5.0 (Adobe Systems, Mountain View, Calif.). Final images were printed with a NP-1600M Medical Color Printer (Codonics, Inc., Middleburg Heights, Ohio). To determine the proportion of cells exhibiting hyphal or yeast growth morphology, four areas with 200 cells each were counted on two separate days (total of 1600 cells), and the standard errors of the means calculated.

An investigation TMPN's in vitro antineoplastic activity was also conducted. This investigation included an analysis of both cell growth, and of the effects of TMPN upon tubulin. Inhibition of cancer cell growth was assessed using the Sulforhodamine B assay as previously described [11]. Briefly, cells in 5% fetal calf serum/RPMI-1640 were inoculated into 96 well plates, incubated 24 h and 10-fold dilutions of TMPN added. After a 48 h incubation, plates were fixed with trichloroacetic acid, washed, stained with Sulforhodamine B and read with an automated microtiter plate reader.

Electrophoretically homogeneous bovine brain tubulin [12] was used in studies to evaluate the effects of TMPN on in vitro tubulin polymerization and the binding of [¹³H]colchicine (Dupont-NEN) to tubulin. These studies were performed as described previously [13]. In the polymerization assay, varying drug concentrations were added to 1 mg/ml tubulin to determine the amount of drug that would inhibit the extent of the reaction by 50% (20 min incubation at 30° C.) (IC₅₀ value). In the colchicine binding assay, the effect of varying drug concentrations on the binding of 2 μg/ml colchicine to 100 μg/ml tubulin was measured after 10 min at 37° C. (control reaction about 50% complete).

The results obtained may be summarized as follows. In disk diffusion assays, the synthetic compound TMPN inhibited the growth of yeasts and certain bacteria, primarily gram-positive bacteria as shown in Table 1. However, in broth macrodilution assays, MICs for all bacteria were >64 μg/ml [single exception was N. gonorrhoeae with an MIC of 4 μg/ml. Broth macrodilution assays revealed that TMPN had broad-spectrum antifungal activity. (Table 2) MFC/MIC ratios were <2 for 96% of C. neoformans clinical isolates, 71% of C. albicans clinical isolates and 70% of C. krusei clinical isolates.

TABLE 1
Antimicrobial activities of 1-(3′,4′,5′-trimethoxyphenyl)-
2-nitro-ethylene in the disk diffusion assay

| Organism | MIC (μg/disk) |
|---|---|
| Staphylococcus aureus ATCC 29213 | 3.12-6.25 |
| Staphylococcus epidermidis Presque Isle 4653 | 50-100 |
| Enterococcus faecalis ATCC 29212 | 50-100 |
| Streptococcus pneumoniae ATCC 6303 | 3.12-6.25 |
| Micrococcus luteus Presque Isle 456 | 50-100 |
| Bacillus subtilis Presque Isle 620 | 50-100 |
| Stenotrophomonas maltophilia ATCC 13637 | >100 |
| Pseudomonas aeruginosa Presque Isle 99 | >100 |
| Escherichia coli ATCC 25922 | >100 |
| Neisseria gonorrhoeae ATCC 49226 | 0.39-0.78 |
| Enterobacter cloacae ATCC 13047 | >100 |
| Klebsiella pneumoniae Presque Isle 344 | >100 |
| Proteus vulgaris Presque Isle 365 | 12.5-25 |
| Cryptococcus neoformans ATCC 90112 | 0.78-1.56 |
| Candida albicans ATCC 90028 | 6.25-12.5 |

TABLE 2
Broth macrodilution MICs and MFCs of 1-(3′,4′,5′-trimethoxyphenyl)-
2-nitro-ethylene for reference strains and clinical isolates

| | MIC (μg/ml) | | | MFC (μg/ml) | | |
|---|---|---|---|---|---|---|
| Organism (no. of strains) | Range | 50%^{a} | 90%^{a} | Range | 50%^{b} | 90%^{b} |
| Fluconazole-resistant Cryptococcus neoformans (4) | 4-8 | | | 4-16 | | |
| C. neoformans (24) | 2-16 | 4 | 8 | 4-32 | 8 | 16 |
| C. neoformans ATCC 90112 | 2 | | | 4 | | |
| C. neoformans ATCC 66031 | 2 | | | 4 | | |
| C. neoformans ATCC 14116 | 8 | | | 16 | | |
| C. neoformans ATCC 32045 | 4 | | | 4 | | |
| C. ater ATCC 14247 | 8 | | | 16 | | |
| C. uniguttulatus ATCC 66033 | 16 | | | 32 | | |
| C. uniguttulatus ATCC 34143 | 8 | | | 16 | | |
| C. laurentii ATCC 66036 | 16 | | | 32 | | |
| C. laurentii ATCC 34142 | 32 | | | 32 | | |
| C. laurentii ATCC 18803 | 16 | | | 32 | | |
| C. albidus ATCC 66030 | 8 | | | 16 | | |
| C. albidus ATCC 40666 | 8 | | | 32 | | |
| C. albidus ATCC 34140 | 32 | | | 32 | | |
| Filobasidium uniguttulatum ATCC 24227 | 0.5 | | | 2 | | |
| Candida albicans (7) | 4-32 | | | 4->64 | | |
| Ketoconazole-resistant C. albicans ATCC 64124 | 16 | | | 32 | | |
| Flucytosine-resistant C. albicans ATCC 32354 | 16 | | | 16 | | |
| C. albicans ATCC 90028 | 32 | | | 32 | | |
| C. albicans ATCC 10231 | 8 | | | 8 | | |
| C. albicans ATCC 14053 | 16 | | | 16 | | |
| C. albicans ATCC 60193 | 16 | | | 16 | | |
| C. parapsilosis (10) | 16-32 | | | 32->64 | | |
| C. parapsilosis ATCC 22019 | 16 | | | 32 | | |
| Amphotericin B-resistant C. lusitaniae ATCC 42720 | 16 | | | 16 | | |
| C. glabrata (8) | 8-32 | | | 16->64 | | |
| C. glabrata ATCC 90030 | 4 | | | 16 | | |
| C. glabrata ATCC 2001 | 8 | | | 16 | | |
| C. guilliermondii (9) | 64->64 | | | >64 | | |
| C. krusei (10) | 8-16 | 8 | 16 | 16-64 | 32 | 32 |
| C. rugosa (7) | 1-64 | | | 8->64 | | |
| C. tropicalis (9) | 32-64 | | | 32->64 | | |
| C. utilis ATCC 22023 | 8 | | | 8 | | |
| C. utilis ATCC 9226 | 4 | | | 8 | | |
| C. viswanathii ATCC 22981 | 32 | | | >64 | | |
| Rhodotorula mucilaginosa ATCC 9449 | 4 | | | 8 | | |
| Kluyveromyces marxianus ATCC 365534 | 4 | | | 4 | | |
| K. apiculate ATCC 9774 | 1 | | | 2 | | |
| Trichosporon cutaneum ATCC 28592 | 4 | | | 8 | | |
| T. inkin ATCC 18020 | 8 | | | 16 | | |
| T. asahii ATCC 20039 | 8 | | | 16 | | |
| T. mucoides ATCC 90046 | 16 | | | 32 | | |
| T. ovoides ATCC 90040 | 16 | | | 32 | | |
| Blastoschizomyces capitalus ATCC 10663 | 8 | | | 32 | | |
| Epidermophyton floccosum ATCC 52066 | 2 | | | ND | | |
| Paecilomyces lilacinus (1) | 32 | | | 64 | | |
| Rhizopus oligosporus ATCC 22959 | 8 | | | >64 | | |
| R. nigracans (ASU culture collection) | 32 | | | >64 | | |
| Aspergillus fumigatus ATCC 96918 | 8 | | | >64 | | |
| A. nidulans strain FGSC4^{b} | 8 | | | 16 | | |
| A. flavus (ASU culture collection) | 16 | | | >64 | | |
| A. niger (ASU culture collection) | 16 | | | 64 | | |

^{a}50% and 90%, MICs at which 50 and 90% of the strains, respectively, are inhibited.
^{b}50% and 90%, MFCs at which 50% and 90% of the strains, respectively, are killed.

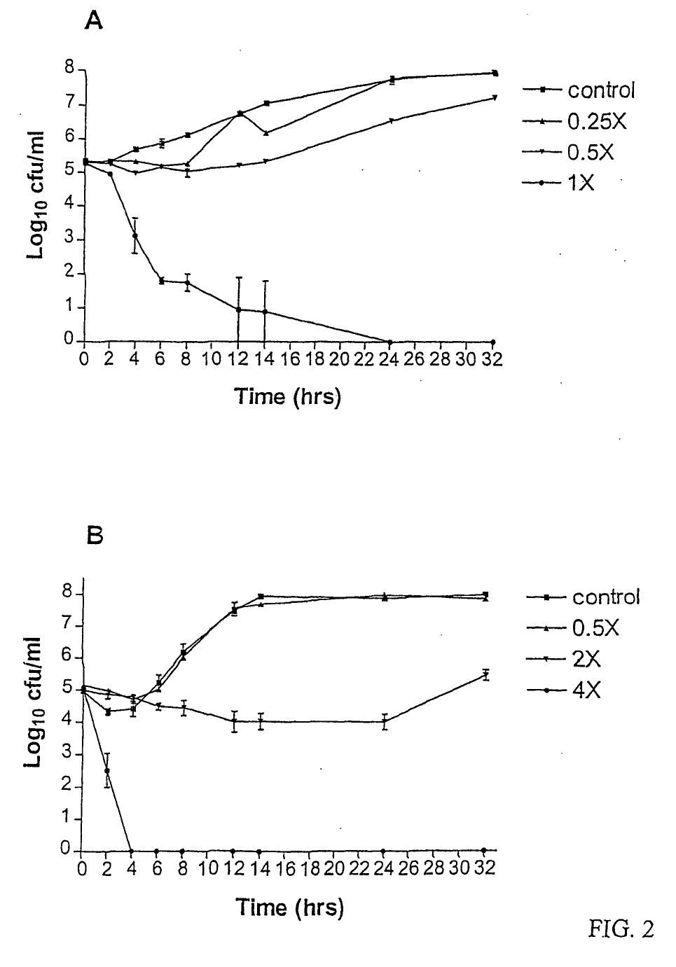

When data for all 60 Candida spp. clinical isolates were combined, 47% had MFC/MIC ratios ≦2, and 60% had MFC/MIC ratios ≦4. MICs and MFCs were identical or differed by no more than a single 2-fold dilution when broth macrodilution assays were performed at pH 5, pH 6 and pH 7 (Table 3). The compound was not active against all species in the presence of 50% human serum, and serum inactivation did not appear to be due to serum albumin binding or serum agglutinins. The frequency of occurrence of single-step resistant mutants at four times the broth macrodilution MIC was ≦10⁻⁶ for the three strains tested, C. neoformans (ATCC 90112), C. albicans (ATCC 90028) and T. inkin (ATCC 18020). FIG. 2 summarizes the time-kill curves for C. neoformans (ATCC 90112) (FIG. 2A) and C. albicans (ATCC 90028) (FIG. 2B). For C. neoformans, time to 99.9% kill was between 4 and 6 h at the MIC. For C. albicans, time to 99.9% kill was between 2 and 4 h at four times the MIC.

TABLE 3
Effect of pH, human serum or bovine serum
albumin on MICs and MFCs of
1-(3′,4′,5′-trimethoxyphenyl)-2-nitro-ethylene

| | | MIC (MFC) |
|---|---|---|
| Organism | Treatment | in μg/ml |
| Cryptococcus neoformans | pH 5 | 2 (4), 1 (2)^{a}. |
| ATCC 90112 | pH 6 | 4 (4), 4 (4) |
| | pH 7 | 2 (4), 2 (4) |
| | no serum | 2 (4) |
| | 50% human serum | >64 |
| | no bovine serum albumin | 2 (4) |
| | 20 μg/ml bovine serum albumin | 4 (4) |
| | 40 μg/ml bovine serum albumin | 4 (4) |
| Candida albicans | pH 5 | 32 (32), 32 (32) |
| ATCC 90028 | pH 6 | 32 (32), 32 (32) |
| | pH 7 | 32 (32), 32 (32) |
| | no serum | 32 (32) |
| | 50% human serum | >64 |
| | no bovine serum albumin | 32 (32) |
| | 20 μg/ml bovine serum albumin | 32 (32) |
| | 40 μg/ml bovine serum albumin | 16 (32) |
| Trichosporon inkin ATCC 18020 | pH 5 | 4 (8), 4 (8) |
| | pH6 | 8 (16), 8 (16) |
| | pH7 | 8 (16), 8 (16) |
| | no serum | 8 (16) |
| | 50% human serum | >64 |
| Aspergillus fumigatus | pH 5 | 4 (32), 8 (>64) |
| ATCC 96918 | pH 6 | 4 (32), 8 (>64) |
| | pH 7 | 4 (32), 8 (>64) |
| | no serum | 8 (16) |
| | 50% human serum | 16 (>64) |

^{a}repeat experiment

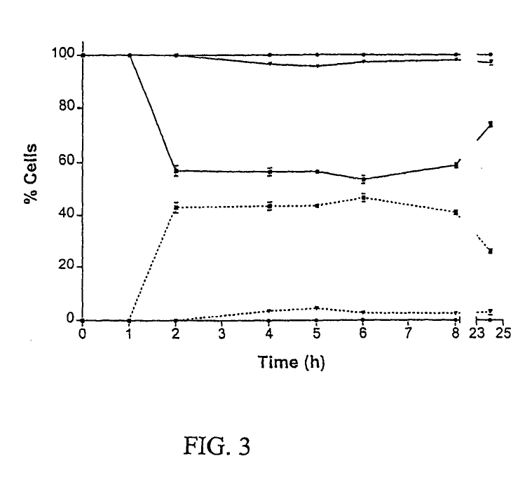

Video-enhanced DIC optics were used to investigate possible morphological alterations in drug-treated C. albicans (ATCC 90028). Cultures were exposed to varying concentrations of TMPN or an equivalent concentration of DMSO (controls), and samples removed late log-phase for microscopy (FIGS. 3,4). From 2-8 h, cultures treated with DMSO alone had approximately the same number of cells with buds as cells with hyphal extensions. Although C. albicans grew at the same rate as controls when exposed to one half times the TMPN MIC, cells with hyphal extensions were not observed in one half times the MIC-treated cultures. Hyphae were rarely seen in one quarter times the MIC-treated cultures, and remained <20 μm in length.

TMPN inhibited the growth of the murine P388 lymphocytic leukemia cell line and six human cancer cell lines, (Table 4) with GI₅₀ values ranging from 1.1-4.1 μg/ml. For inhibition of tubulin polymerization, TMPN was compared with the potent colchicine binding site agent combretastatin A-4. TMPN had an IC₅₀=0.60±0.07 (S.D.) μg/ml for inhibition of the extent of assembly (20 min incubation at 30° C.) versus 0.32+0.02 μg/ml for combretastatin A-4. Combretastatin A-4 at 1.6 μg/ml (5 μM) inhibited [³H]colchicine binding to tubulin by 98+1%, while TMPN at 1.2 μg/ml (5 μM) was minimally inhibitory (13±0.5%). However, when the TMPN concentration was raised to 12 μg/ml (50 uM), there was 69±0.5% inhibition.

TABLE 4
Inhibition of murine P388 lymphocytic
leukemia and human cancer cell line
growth by 1-(3′,4′,5′-trimethoxyphenyl)-2-nitro-ethylene

| Cell line | GI₅₀^{a} (μg/ml) |
|---|---|
| P388 leukemia | 4.15 |
| Pancreas BXPC-3 | 1.6 |
| Ovarian OVCAR-3 | 1.8 |
| CNS SF-295 | 2.0 |
| Lung-NSC NCI-H460 | 1.4 |
| Colon KM20L2 | 1.4 |
| Prostate DU-145 | 1.1 |

^{a}GI₅₀ , inhibition of 50% of cell growth.

Based upon the foregoing observations, these compositions are believed useful in the treatment of one or more fungal infections, such as Aspergillosis, Candidiasis or thrush, internal infections such as cryptococcosis, epidermal infections, infections caused by antibiotic resistant fungi and the like. Similar fungal infections are enumerated in the AMA Home Medical Encyclopedia published by Random House, Inc. 1989.

The dosage administered will be dependent upon the identity of the fungus; the location of the fungal infection; the type of host involved; the nature of concurrent treatment, if any; and the frequency of treatment specified.

Illustratively, dosage levels of the administered active ingredients are: intravenous, 0.1 to about 200 .mu.g/kg; orally, 5 to about 1000 mu.g/kg of host body weight. Expressed in terms of concentration, an active ingredient can be present in the compositions of the present invention for localized use about the cutis, intranasally, pharyngolaryngeally, bronchially, intravaginally, or ocularly in a concentration of from about 0.01 to about 50% w/w of the composition; preferably about 1 to about 20% w/w of the composition; and for parenteral use in a concentration of from about 0.05 to about 50% w/v of the composition and preferably from about 5 to about 20% w/v.

The compositions of the present invention are preferably presented for administration to humans and animals in salves and ointments for topical application although unit dosage forms, such as tablets, capsules, pills, powders, suppositories, sterile parenteral solutions or suspensions, sterile non-parenteral solutions or suspensions, lozenges and the like, containing suitable quantities of an active ingredient.

For oral administration either solid or fluid unit dosage forms can be prepared. Powders are prepared quite simply by comminuting the active ingredient to a suitably fine size and mixing with a similarly comminuted diluent. The diluent can be an edible carbohydrate material such as lactose or starch. Advantageously, a sweetening agent or sugar is present as well as a flavoring oil. Preparing a powder mixture as hereinbefore described and filling into formed gelatin sheaths produces capsules. Advantageously, as an adjuvant to the filling operation, a lubricant such as talc, magnesium stearate, calcium stearate and the like is added to the powder mixture before the filling operation.

Soft gelatin capsules are prepared by machine encapsulation of a slurry of active ingredients with an acceptable vegetable oil, light liquid petrolatum or other inert oil or triglyceride.

Tablets are made by preparing a powder mixture, granulating or slugging, adding a lubricant and pressing into tablets. The powder mixture is prepared by mixing an active ingredient, suitably comminuted, with a diluent or base such as starch, lactose, kaolin, dicalcium phosphate and the like. The powder mixture can be granulated by wetting with a binder such as corn syrup, gelatin solution, methylcellulose solution or acacia mucilage and forcing through a screen. As an alternative to granulating, the powder mixture can be slugged, i.e., run through the tablet machine and the resulting imperfectly formed tablets broken into pieces (slugs). The slugs can be lubricated to prevent sticking to the tablet-forming dies by means of the addition of stearic acid, a stearic salt, talc or mineral oil. The lubricated mixture is then compressed into tablets.

Advantageously, the tablet can be provided with a protective coating consisting of a sealing coat or enteric coat of shellac, a coating of sugar and methylcellulose and polish coating of carnauba wax.

Fluid unit dosage forms for oral administration such as in syrups, elixirs and suspensions can be prepared wherein each teaspoonful of composition contains a predetermined amount of an active ingredient for administration. The water-soluble forms can be dissolved in an aqueous vehicle together with sugar, flavoring agents and preservatives to form a syrup. An elixir is prepared by using a hydroalcoholic vehicle with suitable sweeteners together with a flavoring agent. Suspensions can be prepared of the insoluble forms with a suitable vehicle with the aid of a suspending agent such as acacia, tragacanth, methylcellulose and the like.

For parenteral administration, fluid unit dosage forms are prepared utilizing an active ingredient and a sterile vehicle, water being preferred. The active ingredient, depending on the form and concentration used, can be either suspended or dissolved in the vehicle. In preparing solutions the active ingredient can be dissolved in a suitable vehicle for injection and filter sterilized before filling into a suitable vial or ampule and sealing. Advantageously, adjuvants such as a local anesthetic, preservative and buffering agents can be dissolved in the vehicle.

Parenteral suspensions are prepared in substantially the same manner except that an active ingredient is suspended in the vehicle instead of being dissolved and sterilization cannot be accomplished by filtration. The active ingredient can be sterilized by exposure to ethylene oxide before suspending in the sterile vehicle. Advantageously, a surfactant or wetting agent is included in the composition to facilitate uniform distribution of the active ingredient.

In addition to oral and parenteral administration, the vaginal routes can be utilized particularly by means of a suppository. A vehicle which has a melting point at about body temperature or one that is readily soluble can be utilized. For example, cocoa butter and various polyethylene glycols (Carbowaxes) can serve as the vehicle.

For use as aerosols, the active ingredients can be packaged in a pressurized aerosol container together with a gaseous or liquefied propellant, for example, dichlorodifluoromethane, carbon dioxide, nitrogen, propane, and the like, with the usual adjuvants such as cosolvents and wetting agents, as may be necessary or desirable.

In a preferred practice for the treatment of dermatological fingi, the active ingredient will be delivered to the site as an ointment or salve that will comprise water and oil emulsion as the principal carrier. Other conventional ingredients, when conditions and aesthetics dictate, include petrolatum and mineral oil, lipophilic solubilizers such as polyethylene glycol, carbowax, moisturizers such as lanolin and fragrance.

The term “unit dosage form” as used in the specification and claims refers to physically discrete units suitable as unitary dosages for human and animal subjects, each unit containing a predetermined quantity of active material calculated to produce the desired therapeutic effect in association with the required pharmaceutical diluent, carrier or vehicle. The specifications for the novel unit dosage forms of this invention are dictated by and are directly dependent on (a) the unique characteristics of the active material and the particular therapeutic effect to be achieved, and (b) the limitation inherent in the art of compounding such an active material for therapeutic use in humans, as disclosed in this specification, these being features of the present invention. Examples of suitable unit dosage forms in accord with this invention are tablets, capsules, troches, suppositories, powder packets, wafers, cachets, teaspoonfuls, tablespoonfuls, dropperfuls, ampules, vials, segregated multiples of any of the foregoing, and other forms as herein described.

The active ingredient to be employed as an antifingal agent can be easily prepared in such unit dosage form with the employment of pharmaceutical materials which themselves are available in the art and can be prepared by established procedures. The following preparations are illustrative of the preparation of the unit dosage forms of the present invention, and not as a limitation thereof. Several dosage forms were prepared embodying the present invention. They are shown in the following examples in which the notation “active ingredient” signifies TMPN, or a close homolouge, inclusive.

Composition “A”

Hard-Gelatin Capsules

One thousand two-piece hard gelatin capsules for oral use, each capsule containing 200 .mu.g of an active ingredient are prepared from the following types and amounts of ingredients:

| | |
|---|---|
| Active ingredient, micronized | 200 g |
| Corn Starch | 20 g |
| Talc | 20 g |
| Magnesium stearate | 2 g |

The active ingredient, finely divided by means of an air micronizer, is added to the other finely powdered ingredients, mixed thoroughly and then encapsulated in the usual manner. The foregoing capsules are useful for treating a fungal disease by the oral administration of one or two capsules one to four times a day.

Using the procedure above, capsules are similarly prepared containing an active ingredient in 50, 250 and 500 mu.g amounts by substituting 50 .mu.g, 250 .mu.g and 500 .mu.g of an active ingredient for the 200 .mu.g used above.

Composition “B”

Soft Gelatin Capsules

One-piece soft gelatin capsules for oral use, each containing 200 .mu.g of an active ingredient, finely divided by means of an air micronizer, are prepared by first suspending the compound in 0.5 ml of corn oil to render the material capsulatable and then encapsulating in the above manner.

The foregoing capsules are useful for treating a fungal disease by the oral administration of one or two capsules one to four times a day.

Composition “C”

Tablets

One thousand tablets, each containing 200 .mu.g of an active ingredient, are prepared from the following types and amounts of ingredients:

| | |
|---|---|
| Active ingredient, micronized | 200 g |
| Lactose | 300 g |
| Corn starch | 50 g |
| Magnesium stearate | 4 g |
| Light liquid petrolatum | 5 g |

The active ingredient, finely divided by means of an air micronizer, is added to the other ingredients and then thoroughly mixed and slugged. The slugs are broken down by forcing them through a Number Sixteen screen. The resulting granules are then compressed into tablets, each tablet containing 200 .mu.g of the active ingredient.

The foregoing tablets are useful for treating a fungal disease by the oral administration of one or two tablets one to four times a day. Using the procedure above, tablets are similarly prepared containing an active ingredient in 250 .mu.g and 100 .mu.g amounts by substituting 250 .mu.g and 100 .mu.g of an active ingredient for the 200 .mu.g used above.

Composition “D”

Oral Suspension

One liter of an aqueous suspension for oral use, containing in each teaspoonful (5 ml) dose, 50 .mu.g of an active ingredient, is prepared from the following types and amounts of ingredients:

| | | |
|---|---|---|
| Active ingredient, micronized | 10 | g |
| Citric acid | 2 | g |
| Benzoic acid | 1 | g |
| Sucrose | 790 | g |
| Tragacanth | 5 | g |
| Lemon Oil | 2 | g |
| Deionized water, | q.s. 1000 | ml |

The citric acid, benzoic acid, sucrose, tragacanth and lemon oil are dispersed in sufficient water to make 850 ml of suspension. The active ingredient, finely divided by means of an air micronizer, is stirred into the syrup unit uniformly distributed. Sufficient water is added to make 1000 ml. The composition so prepared is useful for treating a fungal disease at a dose of 1 teaspoonful (15 ml) three times a day.

Composition “E”

Parenteral Product

One liter of a sterile aqueous suspension for parenteral injection, containing 30. mu.g of an active ingredient in each milliliter for treating a fungal disease, is prepared from the following types and amounts of ingredients:

| | | |
|---|---|---|
| Active ingredient, micronized | 30 | g |
| POLYSORBATE 80 | 5 | g |
| Methylparaben | 2.5 | g |
| Propylparaben | 0.17 | g |
| Water for injection, | q.s. 1000 | mi. |

All the ingredients, except the active ingredient, are dissolved in the water and the solution sterilized by filtration. To the sterile solution is added the sterilized active ingredient, finely divided by means of an air micronizer, and the final suspension is filled into sterile vials and the vials sealed. The composition so prepared is useful for treating a fungal disease at a dose of 1 milliliter (1 ml) three times a day.

Composition “F”

Vaginal Suppository

One thousand suppositories, each weighing 2.5 g and containing 200 .mu.g of an active ingredient are prepared from the following types and amounts of ingredients:

| | |
|---|---|
| Active ingredient, micronized | 15 g |
| Propylene glycol | 150 g |
| Polyethylene glycol #4000, q.s. | 2,500 g |

The active ingredient is finely divided by means of an air micronizer and added to the propylene glycol and the mixture passed through a colloid mill until uniformly dispersed. The polyethylene glycol is melted and the propylene glycol dispersion is added slowly with stirring. The suspension is poured into unchilled molds at 40.degree. C. The composition is allowed to cool and solidify and then removed from the mold and each suppository foil wrapped. The foregoing suppositories are inserted vaginally for treating candidiasis (thrush).

Composition “G”

Intranasal Suspension

One liter of a sterile aqueous suspension for intranasal instillation, containing 20 .mu.g of an active ingredient in each milliliter, is prepared from the following types and amounts of ingredients:

| | | |
|---|---|---|
| Active ingredient, micronized | 15 | g |
| POLYSORBATE 80 | 5 | g |
| Methylparaben | 2.5 | g |
| Propylparaben | 0.17 | g |
| Delonized water, | q.s. 1000 | ml. |

All the ingredients, except the active ingredient, are dissolved in the water and the solution sterilized by filtration. To the sterile solution is added the sterilized active ingredient, finely divided by means of an air micronizer, and the final suspension is aseptically filled into sterile containers.

The composition so prepared is useful for treating a fungal disease, by intranasal instillation of 0.2 to 0.5 ml given one to four times per day. An active ingredient can also be present in the undiluted pure form for use locally about the cutis, intranasally, pharyngolaryngeally, bronchially, or orally.

Composition “H”

Powder

Five grams of an active ingredient in bulk form is finely divided by means of an air micronizer. The micronized powder is placed in a shaker-type container. The foregoing composition is useful for treating a fungal disease, at localized sites by applying a powder one to four times per day.

Composition “I”

Oral Powder

One hundred grams of an active ingredient in bulk form are finely divided by means of an air micronizer. The micronized powder is divided into individual doses of 200 .mu.g and packaged. The foregoing powders are useful for treating a fungal disease, by the oral administration of one or two powders suspended in a glass of water, one to four times per day.

Composition “J”

Insufflation

One hundred grams of an active ingredient in bulk form are finely divided by means of an air micronizer. The foregoing composition is useful for treating a fungal disease, by the inhalation of 300 .mu.g one to four times a day.

Composition “K”

Ointment

One hundred grams of an active ingredient in bulk form are finely divided by means of an air micronizer. The micronized powder is them admixed into a water and oil emulsion with the addition of suitable moisturizers and fragrances as desired. The foregoing ointment is useful for treating a fungal disease by one topical application of the ointment on the affected area as needed, preferably at least twice a day.

From the foregoing, it becomes readily apparent that a new and useful antifingal agent and new and useful antifungal preparations have been herein described and illustrated which fulfill the aforestated object in a remarkably unexpected fashion. It is, of course, understood that such modifications, alterations and adaptations as will readily occur to the artisan confronted with this disclosure are intended within the spirit of the present invention which is limited only by the scope of the claims appended hereto

1. A method of treating a host afflicted by a fungi (this includes yeast and filamentous forms) by administering an effective amount of 1-(3′,4,′5′-trimethoxyphenyl)-2-nitro-ethylene thereto.

2. The method according to claim 1 wherein said administration is systemic.

3. The method according to claim 1 wherein said administration is topical.

4. A method of using an effective amount of 1-(3′,4,′5′-trimethoxyphenyl)-2-nitro-ethylene as a biochemical probe.

5. A method according to claim 4 wherein said probe is used to investigate fungal dimorphism.
